SEQUENCE LISTING (7 sequences)

SEQ ID NO: 1 - protein, 182 aa, Bacillus cibi [sequence omitted]

SEQ ID NO: 2 - protein, 182 aa, Artificial Sequence
  Variant
IPPGTPSKSAAQYQLNALTVKPEGPMLGYSRDLFPHWIPQGGGCDTRQVVLKRDADWYVGNCPVTSGSWYSYYDGLYFTNPSDLDIDHIVPLAEAWRSGASSWTTSKRRDFANDLDGPQLIAVSASTNRSKGDQDPSTWQPPRPGAHCGYSKWWISTKYKWGLSLQLSEKTALQDMLNSCLY

SEQ ID NO: 3 - protein, 203 aa, Morchella costata
MKLTAVALFFTTALAAPTLEKRTPPNIPTAASANTMLAALTTRTTDATGYSRDLFPHWITQSGSCNTREVVLARDGSNVVQASDCSASSGTWFSPYDGATWTAASDLDIDHVVPLSDAWKSGANTWTTAGRQAFANDLTNPQLIAVTDNVNQAKGDKSPDAWKPPLTSYYCTYARMWVKVKSVYSLSVTSAERSALTSMLNTC

SEQ ID NO: 4 - protein, 213 aa, Urnula sp
MKFSTVLPLVFTLVAGAPAPTPIVDQAAIEKRLPTGIPTAATAKTLLAGLGTRTTDATGYDRDLFPHWITISGNCNTRETVLNRDGTNLDIGTDCYPDSGTWVSPYDGATWTAASDVDIDHVVPLSEAWKAGANAWTTAQRQAFANDLTNPQLVAVTDNVNSAKGDKTPDLWKPPLTSFHCTYARMYVKVKSVYSLTVKSAERTALTSMLATC

SEQ ID NO: 5 - protein, 207 aa, Neosartorya massa
MTRLLLAALLGTSLVTAIPAPVALPTPPGIPSAATAESELAALTVAAQGSSSGYSRDLFPHWISQGGSCNTREVVLARDGSGVVKDSNCYPTSGSWYSPYDGATWTQASDVDIDHVVPLANAWRSGASKWTTSQRQAFANDLTNPQLMAVTDNVNQAKGDDGPEAWKPPLTSYYCTYAKMWVRVKYVYDLTITSAEKSALVSMLDTC

SEQ ID NO: 6 - protein, 495 aa, Microdochium nivale
MRSAFILALGLITASADALVTRGAIEACLSAAGVPIDIPGTADYERDVEPFNIRLPYIPTAIAQTQTTAHIQSAVQCAKKLNLKVSAKSGGHSYASFGFGGENGHLMVQLDRMIDVISYNDKTGIAHVEPGARLGHLATVLNDKYGRAISHGTCPGVGISGHFAHGGFGFSSHMHGLAVDSVVGVTVVLADGRIVEASATENADLFWGIKGAGSNFGIVAVWKLATFPAPKVLTRFGVTLNWKNKTSALKGIEAVEDYARWVAPREVNFRIGDYGAGNPGIEGLYYGTPEQWRAAFQPLLDTLPAGYVVNPTTSLNWIESVLSYSNFDHVDFITPQPVENFYAKSLTLKSIKGDAVKNFVDYYFDVSNKVKDRFWFYQLDVHGGKNSQVTKVTNAETAYPHRDKLWLIQFYDRYDNNQTYPETSFKFLDGWVNSVTKALPKSDWGMYINYADPRMDRDYATKVYYGENLARLQKLKAKFDPTDRFYYPQAVRPVK

SEQ ID NO: 7 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

(19) United States
(12) Patent Application Publication
(10) Pub. No.: US 2024/0279875 A1
(43) Pub. Date: Aug. 22, 2024

(54) METHOD FOR CONTROLLING SLIME IN A PULP OR PAPER MAKING PROCESS

(71) Applicant: Novozymes A/S, Bagsvaerd (DK)

(72) Inventors: Pedro Emanuel Garcia Loureiro, Vaerlose (DK); Anne Marie Scharff-Poulsen, Vaerlose (DK); Kasper Bay Tingsted, Blovstroed (DK)

(73) Assignee: Novozymes A/S, Bagsvaerd (DK)

(21) Appl. No.: 18/570,301

(22) PCT Filed: Jun. 15, 2022
(86) PCT No.: PCT/EP2022/066384

§ 371 Date: Dec. 14, 2023

(30) Foreign Application Priority Data
Jun. 16, 2021 (DK) PA202100639
Jun. 16, 2021 (EP) 21179682.6

(51) Int. Cl. D21H 21/04; C12N 9/04; C12N 9/22; D21H 17/00

(52) U.S. Cl. CPC D21H 21/04; C12N 9/0006; C12N 9/22; C12Y 101/0301; D21H 17/005

(57) ABSTRACT

The present invention pertains to the field of pulp or paper making. More specifically the present invention relates to a method of preventing a build-up of slime or removing slime from a surface contacted with water from a pulp or paper making process. The present invention can control slime in an efficient and environmentally friendly way.

### REFERENCE TO SEQUENCE LISTING

This application contains a Sequence Listing in computer readable form. The computer readable form is incorporated herein by reference.

### FIELD OF THE INVENTION

The present invention pertains to the field of pulp or paper making. More specifically the present invention relates to a method of preventing a build-up of slime or removing slime from a surface contacted with water from a pulp or paper making process.

### BACKGROUND OF THE INVENTION

Most modern paper mills are operating a warm and closed loop water system under neutral or alkaline conditions which provide a good environment for the growth of microorganisms. In pulp mills, the pH and temperature conditions in the process water (white water) circuit of the pulp drying machines are beneficial for the growth of microorganisms. The microbes in the system or process show slime build-up, i.e., surface-attached, growth, and free-swimming, i.e., planktonic, growth. Slime can develop on the surfaces of a process equipment and can fall off the surfaces. It can reduce water flow; block devices such as filters, wires, or nozzles; deteriorate the final product quality, e.g., by causing holes or colored spots in the final product; or increase downtime due to the need for cleaning or due to breaks in the process. It is difficult to remove the slime from the surfaces of the process equipment and it often requires the use of very strong chemicals. Controlling slime-forming microorganisms by applying toxic biocides is becoming increasingly unacceptable due to environmental concerns and safety. For example, biocides constitute toxicants in the system, and lead to pollution problems consequently. Planktonic microbes may be efficiently controlled by the biocides; however, the use of biocides has not solved all slime problems in paper or board industry, since microorganisms growing in slime are generally more resistant to biocides than the planktonic microbes. In addition, the efficacy of the toxicants is minimized by the slime itself, since the extracellular polysaccharide matrix embedding the microorganisms hinders penetration of the chemicals. Biocides may induce bacterial sporulation and after the treatment of process waters with biocides, a large number of spores may exist in a final product.

There is a need in the paper industry to control slime deposits in an efficient and environmentally friendly way.

### SUMMARY OF THE INVENTION

The present invention provides a method of preventing a build-up of slime or removing slime from a surface contacted with water from a pulp or paper making process, comprising contacting said water with a DNase. In one embodiment, the method is an efficient and environmentally friendly way to prevent a build-up of slime or remove slime from a surface contacted with the water.

The treatment of water from a pulp or paper making process by contacting it with a DNase can efficiently prevent a build-up of slime or remove slime from a surface contacted with the water. The treatment can further reduce downtime by avoiding the need for cleaning or breaks in the pulp or paper making process; reduce spots or holes in a final product; reduce spores in a final product, reduce blocking of devices such as filters, wires, or nozzles, or partly or totally replace biocides. The treatment is efficient and environmentally friendly.

The present invention also relates to a method of manufacturing pulp or paper, comprising subjecting water from pulp or paper making process to a DNase.

The present invention further relates to use of a DNase in preventing the build-up of slime or removing slime from a surface contacted with water from a pulp or paper making process.

The present invention further relates to a composition for preventing a build-up of slime or removing slime from a surface contacted with water from a pulp or paper making process, comprising a DNase and an additional enzyme; a DNase and a surfactant; or a DNase and an additional enzyme, and a surfactant.

Proteases and polysaccharide degrading enzymes have been described in the literature for slime control in papermaking. In a recent review on the control of microbiological problems in papermaking, it discloses the use of several enzyme classes (Pratima Bajpai, Pulp and Paper Industry: Microbiological Issues in Papermaking Chapter 8.4, 2015 Elsevier Inc, ISBN: 978-0-12-803409-5). The industrial benchmark in use as an enzymatic green technology for microbial control in papermaking is based on protease enzymes which prevent bacteria from attaching to a surface and thus preventing slime build-up (Martin Hubbe and Scott Rosencrance (eds.), Advances in Papermaking Wet End Chemistry Application Technologies, Chapter 10.3, 2018 TAPPI PRESS, ISBN: 978-1-59510-260-7). Our invention based on the use of a DNase enzyme has a completely different mode of action from the use of a protease and it was found to have a highly superior effect in the control of slime when compared to the commercial benchmark protease. At the same protein dosage, the prevention effect of the DNase is improved by at least 10%, for example, about 10-5000%, preferably 15-3000%, more preferably 20-2000% compared to the one achieved by the best-in-class protease.

### DETAILED DESCRIPTION OF THE INVENTION

In one aspect, the present invention provides a method of preventing a build-up of slime or removing slime from a surface contacted with water from a pulp or paper making process, comprising contacting said water with a DNase. In one embodiment, the present invention provides a method of preventing a build-up of slime on water from a pulp or paper making process or preventing a build-up of slime from a surface contacted with water from a pulp or paper making process, comprising contacting said water with a DNase. In another embodiment, the present invention provides a method of removing slime from a surface contacted with water from a pulp or paper making process, comprising contacting said water with a DNase.

Microorganisms, such as bacterium, mycoplasma (bacteria without a cell wall) and certain fungi, secrete a polymeric conglomeration of biopolymers, generally composed of extracellular nucleic acids, proteins, and polysaccharides, that form a matrix of extracellular polymeric substance (EPS). The EPS matrix embeds the cells, causing the cells to adhere to each other as well as to any living (biotic) or non-living (abiotic) surface to form a sessile community of microorganisms referred to as a biofilm, slime layer, or slime, or a deposit of microbial origin. A slime colony can also form on solid substrates submerged in or exposed to an aqueous solution, or form as floating mats on liquid surfaces. Primarily, the microorganisms involved in slime formation are special species of spore-forming and nonspore-forming bacteria, particularly capsulated forms of bacteria which secrete gelatinous substances that envelop or encase the cells. Slime forming microorganisms can include filamentous bacteria, filamentous fungi of the mold type, yeasts, and yeast-like organisms. The pulp or paper making processes contain warm waters (e.g., 45-60 degrees C.) that are rich in biodegradable nutrients and have a beneficial pH (e.g., pH 4-9), thus providing a good environment for the growth of microorganisms.

By contacting water from a pulp or paper making process with a DNase, the present invention provides an efficient and environmentally friendly way to prevent a build-up of slime or remove slime from a surface contacted with the water. The slime mainly comprises a matrix of extracellular polymeric substance (EPS) and slime forming microorganisms.

According to the present invention, the term “a DNase” or “deoxyribonuclease” means a polypeptide with DNase activity that catalyzes the hydrolytic cleavage of phosphodiester linkages in the DNA backbone, thus degrading DNA. Examples of enzymes exhibiting a DNase activity are those covered by enzyme classes EC 3.1.11 to EC 3.1.31, as defined in the recommendations of the Nomenclature Committee of the International Union of Biochemistry and Molecular Biology (IUBMB).

In one aspect, the DNase of the present invention has at least 20%, e.g., at least 40%, at least 50%, at least 60%, at least 70%, at least 80%, at least 90%, at least 95%, or 100% of the DNase activity of the DNase having the amino acid sequence of SEQ ID NO: 1, the amino acid sequence of SEQ ID NO: 2, the mature polypeptide of SEQ ID NO: 3, the mature polypeptide of SEQ ID NO: 4, or the mature polypeptide of SEQ ID NO: 5.

The DNase used according to the present invention is a mature polypeptide exhibiting a DNase activity, which comprises or consists of an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence shown as SEQ ID NO: 1, the amino acid sequence shown as SEQ ID NO: 2, the mature polypeptide of SEQ ID NO: 3, the mature polypeptide of SEQ ID NO: 4, or the mature polypeptide of SEQ ID NO: 5.

The relatedness between two amino acid sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

In an embodiment, the amino acid sequence of the DNase is SEQ ID NO: 1, SEQ ID NO: 2, the mature polypeptide of SEQ ID NO: 3, the mature polypeptide of SEQ ID NO: 4, or the mature polypeptide of SEQ ID NO: 5. The term “mature polypeptide” means a polypeptide in its mature form following N terminal and/or C-terminal processing (e.g., removal of signal peptide). In one embodiment, the mature polypeptide of SEQ ID NO: 3 is amino acids 16-203 of SEQ ID NO: 3. In one embodiment, the mature polypeptide of SEQ ID NO: 4 is amino acids 17-213 of SEQ ID NO: 4. In one embodiment, the mature polypeptide of SEQ ID NO: 5 is amino acids 18-207 of SEQ ID NO: 5. In another embodiment, the DNase is a bacterial or fungal DNase, preferably a Bacillus DNase, a Morchella DNase, a Urnula DNase or Neosartorya DNase; more preferably Bacillus cibi DNase, Morchella costata DNase or Neosartorya massa DNase. In another embodiment, the DNase is a Bacillus cibi DNase, a derivative or a variant thereof. In another embodiment, the DNase is Morchella costata DNase, a derivative or a variant thereof. In another embodiment, the DNase is Urnula DNase, a derivative or a variant thereof. In another embodiment, the DNase is Neosartorya massa DNase, a derivative, or a variant thereof. In yet another embodiment, the DNase is a DNase as disclosed in International patent application no. WO 2019/081724, which is hereby incorporated by reference.

In an embodiment, the number of amino acid substitutions, deletions and/or insertions introduced into the amino acid sequence of SEQ ID NO: 1, the amino acid sequence of SEQ ID NO: 2, the mature polypeptide of SEQ ID NO: 3, the mature polypeptide of SEQ ID NO: 4, or the mature polypeptide of SEQ ID NO: 5 is up to 20, e.g., 1, 2, 3, 4, 5, 6, 7, 8, 9, 10, 11, 12, 13, 14, 15, 16, 17, 18, 19 or 20; or up to 10, e.g., 1, 2, 3, 4, 5, 6, 7, 8, 9, 10; or up to 5. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for a DNase activity to identify amino acid residues that are critical to the activity of the molecule. See also, Hilton et al., 1996, J. Biol. Chem. 271: 4699-4708. The active site of the DNase or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

In a preferred embodiment, the DNase is a DNase variant, which compared to a DNase with SEQ ID NO: 1, comprises one, two or more substitutions selected from the group consisting of: T1I, T1L, T1V, S13Y, T22P, S25P, S27L, S39P, S42G, S42A, S42T, S57W, S57Y, S57F, S59V, S591, S59L, V76L, V761, Q109R, S116D, S116E, T127V, T1271, T127L, S144P, A147H, S167L, S1671, S167V, G175D and G175E, wherein the positions correspond to the positions of SEQ ID NO: 1 (numbering according to SEQ ID NO: 1) wherein the variant has a sequence identity to the polypeptide shown in SEQ ID NO: 1 of at least 60%, at least 70%, at least 80%, at least 85%, at least 90%, at least 95%, at least 97%, at least 98%, at least 99% and wherein the variant has a DNase activity. In a more preferred embodiment, the DNase variant is a variant comprising one or more of the substitution sets selected from the group consisting of: T1I+S13Y, T1I+T22P, T1I+S25P, T1I+S27L, T1I+S39P, T1I+S42G, T1I+S42A, T1I+S42T, T1I+S57W, T1I+S57Y, T1I+S57F, T1I+S59V, T1I+S591, T1I+S59L, T1I+V76L, T1I+V761, T1I+Q109R, T1I+S116D, T1I+S116E, T1I+T127V, T1I+T1271, T1I+T127L, T1I+S144P, T1I+A147H, T1I+S167L, T1I+S1671, T1I+S167V, T1I+G175D, T1I+G175E, T1L+S13Y, T1 L+T22P, T1 L+S25P, T1 L+S27L, T1 L+S39P, T1 L+S42G, T1 L+S42A, T1 L+S42T, T1 L+S57W, T1 L+S57Y, T1 L+S57F, T1 L+S59V, T1 L+S591, T1 L+S59L, T1 L+V76L, T1 L+V761, T1 L+Q109R, T1L+S116D, T1L+S116E, T1L+T127V, T1L+T1271, T1L+T127L, T1L+S144P, T1L+A147H, T1L+S167L, T1L+S1671, T1L+S167V, T1L+G175D, T1L+G175E, T1V+S13Y, T1V+T22P, T1V+S25P, T1V+S27L, T1V+S39P, T1V+S42G, T1V+S42A, T1V+S42T, T1V+S57W, T1V+S57Y, T1V+S57F, T1V+S59V, T1V+S591, T1V+S59L, T1V+V76L, T1V+V761, T1V+Q109R, T1V+S116D, T1V+S116E, T1V+T127V, T1V+T1271, T1V+T127L, T1V+S144P, T1V+A147H, T1V+S167L, T1V+S1671, T1V+S167V, T1V+G175D, T1V+G175E, S13Y+T22P, S13Y+S25P, S13Y+S27L, S13Y+S39P, S13Y+S42G, S13Y+S42A, S13Y+S42T, S13Y+S57W, S13Y+S57Y, S13Y+S57F, S13Y+S59V, S13Y+S591, S13Y+S59L, S13Y+V76L, S13Y+V761, S13Y+Q109R, S13Y+S116D, S13Y+S116E, S13Y+T127V, S13Y+T1271, S13Y+T127L, S13Y+S144P, S13Y+A147H, S13Y+S167L, S13Y+S1671, S13Y+S167V, S13Y+G175D, S13Y+G175E, T22P+S25P, T22P+S27L, T22P+S39P, T22P+S42G, T22P+S42A, T22P+S42T, T22P+S57W, T22P+S57Y, T22P+S57F, T22P+S59V, T22P+S591, T22P+S59L, T22P+V76L, T22P+V761, T22P+Q109R, T22P+S116D, T22P+S116E, T22P+T127V, T22P+T1271, T22P+T127L, T22P+S144P, T22P+A147H, T22P+S167L, T22P+S1671, T22P+S167V, T22P+G175D, T22P+G175E, S25P+S27L, S25P+S39P, S25P+S42G, S25P+S42A, S25P+S42T, S25P+S57W, S25P+S57Y, S25P+S57F, S25P+S59V, S25P+S591, S25P+S59L, S25P+V76L, S25P+V761, S25P+Q109R, S25P+S116D, S25P+S116E, S25P+T127V, S25P+T1271, S25P+T127L, S25P+S144P, S25P+A147H, S25P+S167L, S25P+S1671, S25P+S167V, S25P+G175D, S25P+G175E, S27L+S39P, S27L+S42G, S27L+S42A, S27L+S42T, S27L+S57W, S27L+S57Y, S27L+S57F, S27L+S59V, S27L+S591, S27L+S59L, S27L+V76L, S27L+V761, S27L+Q109R, S27L+S116D, S27L+S116E, S27L+T127V, S27L+T1271, S27L+T127L, S27L+S144P, S27L+A147H, S27L+S167L, S27L+S1671, S27L+S167V, S27L+G175D, S27L+G175E, S39P+S42G, S39P+S42A, S39P+S42T, S39P+S57W, S39P+S57Y, S39P+S57F, S39P+S59V, S39P+S591, S39P+S59L, S39P+V76L, S39P+V761, S39P+Q109R, S39P+S116D, S39P+S116E, S39P+T127V, S39P+T1271, S39P+T127L, S39P+S144P, S39P+A147H, S39P+S167L, S39P+S1671, S39P+S167V, S39P+G175D, S39P+G175E, S42G+S57W, S42G+S57Y, S42G+S57F, S42G+S59V, S42G+S591, S42G+S59L, S42G+V76L, S42G+V761, S42G+Q109R, S42G+S116D, S42G+S116E, S42G+T127V, S42G+T1271, S42G+T127L, S42G+S144P, S42G+A147H, S42G+S167L, S42G+S1671, S42G+S167V, S42G+G175D, S42G+G175E, S42A+S57W, S42A+S57Y, S42A+S57F, S42A+S59V, S42A+S591, S42A+S59L, S42A+V76L, S42A+V761, S42A+Q109R, S42A+S116D, S42A+S116E, S42A+T127V, S42A+T1271, S42A+T127L, S42A+S144P, S42A+A147H, S42A+S167L, S42A+S1671, S42A+S167V, S42A+G175D, S42A+G175E, S42T+S57W, S42T+S57Y, S42T+S57F, S42T+S59V, S42T+S591, S42T+S59L, S42T+V76L, S42T+V761, S42T+Q109R, S42T+S116D, S42T+S116E, S42T+T127V, S42T+T1271, S42T+T127L, S42T+S144P, S42T+A147H, S42T+S167L, S42T+S1671, S42T+S167V, S42T+G175D, S42T+G175E, S57W+S59V, S57W+S591, S57W+S59L, S57W+V76L, S57W+V761, S57W+Q109R, S57W+S116D, S57W+S116E, S57W+T127V, S57W+T1271, S57W+T127L, S57W+S144P, S57W+A147H, S57W+S167L, S57W+S1671, S57W+S167V, S57W+G175D, S57W+G175E, S57Y+S59V, S57Y+S591, S57Y+S59L, S57Y+V76L, S57Y+V761, S57Y+Q109R, S57Y+S116D, S57Y+S116E, S57Y+T127V, S57Y+T1271, S57Y+T127L, S57Y+S144P, S57Y+A147H, S57Y+S167L, S57Y+S1671, S57Y+S167V, S57Y+G175D, S57Y+G175E, S57F+S59V, S57F+S591, S57F+S59L, S57F+V76L, S57F+V761, S57F+Q109R, S57F+S116D, S57F+S116E, S57F+T127V, S57F+T1271, S57F+T127L, S57F+S144P, S57F+A147H, S57F+S167L, S57F+S1671, S57F+S167V, S57F+G175D, S57F+G175E, S59V+V76L, S59V+V761, S59V+Q109R, S59V+S116D, S59V+S116E, S59V+T127V, S59V+T1271, S59V+T127L, S59V+S144P, S59V+A147H, S59V+S167L, S59V+S1671, S59V+S167V, S59V+G175D, S59V+G175E, S591+V76L, S591+V761, S591+Q109R, S591+S116D, S591+S116E, S591+T127V, S591+T1271, S591+T127L, S591+S144P, S591+A147H, S591+S167L, S591+S1671, S591+S167V, S591+G175D, S591+G175E, S59L+V76L, S59L+V761, S59L+Q109R, S59L+S116D, S59L+S116E, S59L+T127V, S59L+T1271, S59L+T127L, S59L+S144P, S59L+A147H, S59L+S167L, S59L+S1671, S59L+S167V, S59L+G175D, S59L+G175E, V76L+Q109R, V76L+S116D, V76L+S116E, V76L+T127V, V76L+T1271, V76L+T127L, V76L+S144P, V76L+A147H, V76L+S167L, V76L+S1671, V76L+S167V, V76L+G175D, V76L+G175E, V761+Q109R, V761+S116D, V761+S116E, V761+T127V, V761+T1271, V761+T127L, V761+S144P, V761+A147H, V761+S167L, V761+S1671, V761+S167V, V761+G175D, V761+G175E, Q109R+S116D, Q109R+S116E, Q109R+T127V, Q109R+T1271, Q109R+T127L, Q109R+S144P, Q109R+A147H, Q109R+S167L, Q109R+S1671, Q109R+S167V, Q109R+G175D, Q109R+G175E, S116D+T127V, S116D+T1271, S116D+T127L, S116D+S144P, S116D+A147H, S116D+S167L, S116D+S1671, S116D+S167V, S116D+G175D, S116D+G175E, S116E+T127V, S116E+T1271, S116E+T127L, S116E+S144P, S116E+A147H, S116E+S167L, S116E+S1671, S116E+S167V, S116E+G175D, S116E+G175E, T127V+S144P, T127V+A147H, T127V+S167L, T127V+S1671, T127V+S167V, T127V+G175D, T127V+G175E, T1271+S144P, T1271+A147H, T1271+S167L, T1271+S1671, T1271+S167V, T1271+G175D, T1271+G175E, T127L+S144P, T127L+A147H, T127L+S167L, T127L+S1671, T127L+S167V, T127L+G175D, T127L+G175E, S144P+A147H, S144P+S167L, S144P+S1671, S144P+S167V, S144P+G175D, S144P+G175E, A147H+S167L, A147H+S1671, A147H+S167V, A147H+G175D, A147H+G175E, S167L+G175D, S167L+G175E, S1671+G175D, S1671+G175E, S167V+G175D and S167V+G175E.

In a further embodiment, the DNase variant is selected from the group consisting of:
i. T1I+S13Y+T22P+S25P+S27L+S39P+S42G+S57W+S59V+V76L+S144P+A147H+S167 L+G175D;
ii. T1I+S13Y+T22P+S27L+S42G+S57W+S59V+V76L+Q109R+S116D+T127V+S144P+A1 47H+S167L+G175D;
iii. T1I+S13Y+T22P+S25P+S27L+S39P+S42G+S57W+S59V+V76L+Q109R+S116D+T127 V+S144P+A147H+S167L+G175D;
iv. T1I+S13Y+T22P+S25P+S27L+S39P+S42G+S57W+S59V+V76L+T77Y+Q109R+S116D+T127V+S144P+A147H+S167L+G175D;
v. T1I+T22P+D561+S57W+V76L+Q109R+S116D+A147H+G162S+S167L+G175N+N178;
vi. T1I+S13Y+T22P+S27L+S39P+S42G+D561+S57W+S59V+V76L+Q109R+S116D+T127 V+S144P+A147H+S167L+G175D;
vii. T1I+T22P+S25P+S27L+S42G+D561+S57Y+S59V+V76L+T77Y+Q109R+S116D+T127V+S144P+A147H+Q166D+S167L+G175D+S181L;
viii. T1I+S13Y+T22P+S25P+S27L+S39P+S42G+D561+S57W+S59V+V76L+Q109R+S116D+T127V+S144P+A147H+S167L+G175D;
ix. T1I+S13Y+T22P+S25P+S27L+S39P+S42G+D561+S57W+S59V+V76L+Q109R+S116D+T127V+S144P+A147H+Q166D+S167L+G175D;
x. T1I+S13Y+T22P+S27R+S39P+S42G+D561+S57W+S59V+V76L+Q109R+S116D+T127 V+S144P+A147H+S167L+G175D;
xi. T1I+S13Y+T22P+S27L+S39P+S42G+D561+S57W+S59V+T65L+V76L+Q109R+S116D+T127V+S144P+A147H+S167L+G175D;
xii. T1I+S13Y+T22P+S27L+L33K+S39P+S42G+D561+S57W+S59V+T65V+V76L+Q109R+S116D+T127V+S144P+A147H+S167L+G175D;
xiii. T1I+S13Y+T22P+S25P+S27R+S39P+S42G+S57W+S59V+S66W+V76L+T77Y+Q109R+S116D+T127V+S144P+A147H+S167L+G175D;
xiv. T1I+S13Y+T22P+S25P+S27L+L33K+S39P+S42G+S57W+S59V+T65V+V76L+T77Y+Q 109R+S116D+T127V+S144P+A147H+S167L+G175D;
xv. T1I+S13Y+T22P+S25P+S27L+L33K+S39P+S42G+S57W+S59V+S66Y+V76L+T77Y+Q 109R+S116D+T127V+S144P+A147H+S167L+G175D;
xvi. T1I+S13Y+T22P+S25P+S27L+S39P+S42G+S57W+S59V+T65V+S66Y+V76L+T77Y+Q 109R+S116D+T127V+S144P+A147H+S167L+G175D;
xvii. T1I+S13Y+T22P+S27L+S39P+S42G+D56L+S57W+S59V+T65V+V76L+Q109R+S1 16D+T127V+S144P+A147H+G162D+S167L+G175D;
xviii. T1I+S13Y+T22P+S27R+S39P+S42G+D56L+S57W+S59V+T65V+V76L+Q109R+S1 16D+T127V+S144P+A147H+S167L+G175D;
xix. T1I+S13Y+T22P+S27R+S39P+S42G+D56L+S57W+S59V+T65V+V76L+Q109R+S116 D+T127V+S144P+A147H+G162D+S167L+G175D;
xx. T1I+S13Y+T22P+S27K+S39P+S42G+D561+S57W+S59V+T65V+V76L+S106L+Q109R+S116D+T127V+S144P+A147H+S167L+G175D;
xxi. T1I+S13Y+T22P+S27K+S39P+S42G+D561+S57W+S59V+T65V+V76L+Q109R+S116D+T127V+S130A+S144P+A147H+S167L+G175D;
xxii. T1I+S13Y+T22P+S27L+S39P+S42G+D56L+S57W+S59V+T65L+V76L+Q109R+S1 16D+T127V+S130A+S144P+A147H+S167L+G175D; and
xxiii. T1I+S13Y+T22P+S27L+S39P+S42G+D561+S57W+S59V+T65V+V76L+Q109R+S11 6D+T127V+S144P+A147H+G162D+S167L+G175.

In a further preferred embodiment, the DNase variant is selected from the group consisting of:
a. T1I+T22P+S57W+V76L+A147H+S167L,
b. T1I+T22P+S57W+V76L+A147H+G175D,
c. T1I+T22P+S57W+V76L+S167L+G175D,
d. T1I+T22P+S57W+A147H+S167L+G175D,
e. T1I+T22P+V76L+A147H+S167L+G175D,
f. T1 I+S57W+V76L+A147H+S167L+G175D,
g. T22P+S57W+V76L+A147H+S167L+G175D, and
h. T1I+T22P+S57W+V76L+A147H+S167L+G175D.

In a further embodiment, the DNase is a DNase variant which compared to the polypeptide of SEQ ID NO: 2 comprises one, two or more substitutions selected from the group consisting of N61D, T651, T65V, S82R, K107Q, T127S, T127V, G149N, S164D and 1181S, wherein the variant has at least 60%, at least 70%, at least 80%, at least 85%, at least 90%, at least 95%, at least 97%, at least 98%, at least 99% sequence identity to SEQ ID NO: 2 and has DNase activity. In a further preferred embodiment, the DNase variant further comprises at least one substitution selected from the group consisting of Q14R, Q14W, K21L, P25S, L33K, Q48D, D561, D56L, S66Y, S68L, Y77T, S102Y, S106A, R109Q, R109T, D116S, D116W, T171W, L181T and L181W. In a further preferred embodiment, the DNase variant comprises a set of substitutions selected from the group consisting of:
a) G149N together with at least one of the substitutions N61D, T651, T65V, S82R, K107Q, T127S, T127V, S164D and L181S;
b) T651 or T65V together with at least two of the substitutions N61 D, S82R, K107Q, T127S, T127V, G149N, S164D and L181S;
c) N61D together with at least two of the substitutions T651/V, S82R, K107Q, T127S/V, G149N, S164D and L181S, preferably at least two of the substitutions T651/V, S82R, K107Q, T127S and S164D;
d) S82R together with at least two of the substitutions N61D, T651, T65V, K107Q, T127S, T127V, G149N, S164D and L181S;
e) K107Q together with at least two of the substitutions N61D, T651, T65V, S82R, T127S, T127V, G149N, S164D and L181S;
f) T127S together with at least two of the substitutions N61D, T651, T65V, S82R, K107Q, G149N, S164D and L181S; and
g) S164D together with at least one of the substitutions N61D, T651, T65V, S82R, K107Q, T127S, T127V, G149N and L181S.

In a further preferred embodiment, the DNase variant comprises a set of substitutions selected from the group consisting of:
K21L+Q48D+T651+S82R+K107Q+T127S;
Q14R+K21L+Q48D+T651+T127S;
Q48D+T651+S82R+T127S+S164D;
N61D+T651+K107Q+T127S+S164D;
Q48D+T651+S82R+K107Q+T127S;
Q14R+N61D+T651+S82R+K107Q;
N61 D+S68L+G149N;
Q14R+N61D+T651+S82R+T127S+S164D;
K21L+Q48D+T651+S82R+K107Q;
Q14R+T651+K107Q+T127S;
N61 D+T651+S82R+T127S+S164D;
K21L+N61D+T651+S82R+K107Q+T127S;
T65V+T127V+G149N;
T651+K107Q+T127S+S164D;
N61D+T651+S82R+K107Q;
Q14R+K21L+N61D+T651+S82R;
Q14R+K21L+N61D+T651+S82R+K107Q;
Q14R+K21L+N61D+T651+T127S;
N61 D+T651+S82R+K107Q+T127S+S164D;
Q14R+K21L+T651+K107Q+T127S;
N61D+T651+K107Q+T127S;
T651+S82R+K107Q+S164D;
K21L+N61D+T651+S82R;
K21L+N61D+T651+T127S;
N61 D+T651+S82R+S164D;
K21L+N61D+T651+S82R+K107Q;
S68L+S106A+G149N;
N61D+T651+T127S+S164D;
Q14R+K21L+N61D+T651;
Q14R+K21L+T651+T127S;
T65V+G149N;
T65V+R109T+T127V;
Q14R+K21L+T651+K107Q;
K21L+T651+S82R+K107Q;
K21L+T651+K107Q+T127S;
N61D+S68L+S102Y+G149N+S164D+L181T;
N61D+S68L+S106A+G149N+S164D;
T65V+R109T+G149N;
T65V+T127V+T171W;
T65V+T127V+L181S;
T651+S164D+L181W;
N61D+S66Y+S102Y+S164D;
N61D+S66Y+S164D;
N61 D+T65V+S164D;
Q14W+N61D+T651;
Q14W+N61D+T651+S164D;
Q14W+N61D+T651+S164D+L181W;
T651+D116W+S164D+L181W;
T651+D116W+S164D;
Q14W+T651+S164D;
R109T+G149N;
G149N+T171W;
G149N;
S164D;
P25S+L33K+D561+T65V+Y77T+T127V+L181S;
P25S+L33K+T65V+Y77T+T127V+L181S;
P25S+L33K+D561+T65V+Y77T+R109Q+T127V+L181S;
P25S+L33K+D561+T65V+Y77T+D116S+T127V+L181S;
D56L+T65V+T127V;
D56L+T65V+T127V+T171W;
T65V+G149N+T171W;
Q48D+T651+K107Q+T127S+S164D;
T65V+Y77T+T127V;
T65V+R109T+T127V+G149N;
T65V+R109T+T171W;
T65V+R109T+G149N+T171W;
P25S+D561+T65V+Y77T+T127V+L181S;
Q14R+K21L+T651+K107Q+T127S;
N61D+S68L+S102Y;
S66Y+T127V+L181S;
N61D+T651+S82R+K107Q+L181D;
T65V+G149N+L181E;
T65V+T127V+G149N+Y182D;
Q48D+N61D+T651+S82R+K107Q;
Q48D+T65V+G149N;
N61D+T651+S82R+T127S+S164D+Y182D;
N61D+T651+S82R+T127S+S164D+L181D;
Q48D+N61D+T651+K107Q+T127S+S164D;
N61D+T651+S82R+T127S+S164D+Y182N;
T651+K107Q+T127S+S164D+Y182D;
N61D+T651+K107Q+T127S+S164D+L181E;
N61 D+T651+K107Q+T127S+S164D+L181D;
T651+K107Q+T127S+S164D+L181T;
T651+K107Q+T127S+S164D+L181E;
N61 D+T651+K107Q+T127S+S164D+Y182D;
T651+K107Q+T127S+S164D+Y182N;
N61D+T651+K107Q+T127S+S164D+L181Q;
N61D+T65V+S164D+Y182N;
N61D+T651+K107Q+T127S+S164D+L181T;
T651+K107Q+T127S+S164D+L181D;
T651+K107Q+T127S+S164D+L181Q;
N61D+T65V+T127S+S164D;
Q48D+N61D+T65V+S164D;
K21 L+N61 D+T651+K107Q+T127S;
Q14W+N61D+T651+R109T+G149N+S164D+L181W;
K21L+N61D+T651+K107Q;
Q14W+N61D+T651+R109T+G149N;
Q14W+T65V+R109T+G149N+W1541+L181W;
Q14W+T65V+R109T+G149N+W1541+S164D;
Q14W+N61D+T65V+R109T+G149N+L181W;
Q14W+T651+R109T+D116W+G149N+S164D+L181W;
T65V+R109T+T127V+T171W; and
R109T+T127V+T171W.

In a further preferred embodiment, the DNase variant comprises a set of substitutions selected from the group consisting of:
T65V+T127V+L181S;
N61D+T651+S82R+K107Q;
T65V+G149N;
N61 D+T651+K107Q+T127S+S164D;
N61D+T651+T127S+S164D;
N61D+T65V+S164D;
T65V+T127V+G149N;
N61D+T651+S82R+T127S+S164D; and
T651+K107Q+T127S+S164D.

The DNase is added in an amount effective to preventing a build-up of slime or removing slime from a surface contacted with water from a pulp or paper making process. In a preferred embodiment, the DNase is added in an amount of 0.001-1000 mg enzyme protein/L, preferably 0.005-500 mg enzyme protein/L, more preferably 0.01 mg-100 mg enzyme protein/L, such as, 0.05 mg-50 mg enzyme protein/L, or 0.1-10 mg enzyme protein/L.

The DNase treatment may be used to control (i.e., reduce or prevent) build-up of slime or remove slime from a surface contacted with water from a pulp or paper making process in any desired environment. In one embodiment, the surface is a solid substrate submerged in or exposed to an aqueous solution, or forms as floating mats on liquid surfaces. In preferred embodiment, the surface is solid surface, for example, a plastic surface or a metal surface. The solid surface can come from a manufacturing equipment, such as surfaces of the pulpers, headbox, machine frame, foils, suction boxes, white water tanks, clarifiers and pipes.

The DNase treatment may be used to control (i.e., reduce or prevent) a build-up of slime or remove slime from a surface contacted with water from a pulp or paper making process. In the present context, the term “water” comprises, but not limited to: 1) cleaning water used to clean a surface in paper-making; 2) process water added as a raw material to the pulp or paper making process; 3) intermediate process water products resulting from any step of the process for manufacturing the paper material; 4) waste water as an output or by-product of the process; 5) water mist in the air, generated by clearing water, process water or waste water at a certain humidity and temperature. In an embodiment, the water is cleaning water, process water, wastewater, and/or water mist in the air. In a particular embodiment, the water is, has been, is being, or is intended for being circulated (re-circulated), i.e., re-used in another step of the process. In a preferred embodiment, the water is process water from recycled tissue production. In a preferred embodiment, the water is process water from liquid packaging board production. In a preferred embodiment, the water is process water from recycled packaging board process. The term “water” in turn means any aqueous medium, solution, suspension, e.g., ordinary tap water, and tap water in admixture with various additives and adjuvants commonly used in pulp or paper making processes. In a particular embodiment the process water has a low content of solid (dry) matter, e.g., below 20%, 18%, 16%, 14%, 12%, 10%, 8%, 7%, 6%, 5%, 4%, 3%, 2% or below 1% dry matter. The water may vary in properties such as pH, conductivity, redox potential and/or ATP. In a preferred embodiment, the water has pH from 4 to 10, conductivity from 100 μS/cm to 12000 μS/cm, redox potential from −500 mV to 1500 mV and/or cellular ATP from 0.1 ng/ml to 1000 ng/ml. In a more preferred embodiment, the water has pH from 5 to 9, conductivity from 1000 μS/cm to 8000 μS/cm, redox potential from −300 mV to 500 mV and/or cellular ATP from 1 ng/ml to 500 ng/ml. In the most preferred embodiment, the water has pH from 6.1 to 7.6, conductivity from 1772 μS/cm to 5620 μS/cm, redox potential from −110 mV to 210 mV and/or cellular ATP from 4.2 ng/ml to 114 ng/ml.

In one embodiment, the pulp or paper making process of the present invention can be carried out separately in a pulp making mill and paper making mill. In a preferred embodiment, the pulp or paper making process is a paper making process which can be carried out in a paper making mill. In another embodiment, the pulp or paper making process is a pulp and paper making process which can be carried out in an integrated paper mill. In an embodiment of the present invention, the process of papermaking starts with the stock preparation, where a suspension of fibers and water is prepared and pumped to the paper machine. This slurry consists of approximately 99.5% water and approximately 0.5% pulp fiber and flows until the “slice” or headbox opening where the fibrous mixture pours onto a traveling wire mesh in the Fourdrinier process, or onto a rotating cylinder in the cylinder. As the wire moves along the machine path, water drains through the mesh while fibers align in the direction of the wire. After the web forms on the wire, the paper machine needs to remove additional water. It starts with vacuum boxes located under the wire which aid in this drainage, then followed by the pressing and drying section where additional dewatering occurs. As the paper enters the press section, it undergoes compression between two rotating rolls to squeeze out more water and then the paper web continues through the steam-heated dryers to lose more moisture. Depending on the paper grade being produced, it will sometimes undergo a sizing or coating process in a second dry-end operation before entering the calendaring stacks as part of the finishing operation. At the end of the paper machine, the paper continues onto a reel for winding to the desired roll diameter. The machine tender cuts the paper at this diameter and immediately starts a new reel. The process is now complete for example in grades of paper used in the manufacture of corrugated paperboard. However, for papers used for other purposes, finishing and converting operations will now occur, typically off-line from the paper machine (Pratima Bajpai, Pulp and Paper Industry: Microbiological Issues in Papermaking, Chapter 2.1, 2015 Elsevier Inc, ISBN: 978-0-12-803409-5).

In one embodiment, fibrous material is turned into pulp and bleached to create one or more layers of board or packaging material, which can be optionally coated for a better surface and/or improved appearance. Board or packaging material is produced on paper machines that can handle higher grammage and several plies.

The temperature and pH for the DNase treatment in the pulp or paper making process is not critical, provided that the temperature and pH is suitable for the enzymatic activity of the DNase. Generally, the temperature and pH will depend on the system, composition or process which is being treated. Suitable temperature and/or pH conditions include 5° C. to 120° C. and/or pH 1 to 12, however, ambient temperatures and pH conditions are preferred. For paper production processes, the temperature and pH will generally be 15° C. to 65° C., for example, 45° C. to 60° C. and pH 3 to 10, for example, pH 4 to 9.

The treatment time will vary depending on, among other things, the extent of the slime problem and the type and amount of the DNase employed. The DNase may also be used in a preventive manner, such that, the treatment time is continuous or carried out a set point in the process.

In a preferred embodiment, the DNase is used to treat water in a pulp or paper making process for manufacturing paper or packaging material. The term “paper or packaging material” refers to paper or packaging material which can be made out of pulp. In an embodiment, the paper and packaging material is selected from the group consisting of printing and writing paper, tissue and towel, newsprint, carton board, containerboard and packaging papers.

The term “pulp” means any pulp which can be used for the production of a paper and packaging material. Pulp is a lignocellulosic fibrous material prepared by chemically or mechanically separating cellulose fibers from wood, fiber crops or waste paper. For example, the pulp can be supplied as a virgin pulp, or can be derived from a recycled source, or can be supplied as a combination of a virgin pulp and a recycled pulp. The pulp may be a wood pulp, a non-wood pulp or a pulp made from waste paper. A wood pulp may be made from softwood such as pine, redwood, fir, spruce, cedar and hemlock or from hardwood such as maple, alder, birch, hickory, beech, aspen, acacia and eucalyptus. A non-wood pulp may be made, e.g., from flax, hemp, bagasse, bamboo, cotton or kenaf. A waste paper pulp may be made by re-pulping waste paper such as newspaper, mixed office waste, computer print-out, white ledger, magazines, milk cartons, paper cups etc.

In other preferred embodiments, the DNase is added in combination (such as, for example, sequentially or simultaneously) with an additional enzyme and/or a surfactant.

Any enzyme having carbohydrate oxidase, lipase, cutinase, protease, pectinase, laccase, peroxidase, cellulase, glucanase, xylanase, mannanase, lysozyme, amylase, glucoamylase, galactanase, and/or levanase activity can be used as additional enzymes in the present invention. Below some non-limiting examples are listed of such additional enzymes. The enzymes written in capitals are commercial enzymes available from Novozymes A/S, Krogshoejvej 36, DK-2880 Bagsvaerd, Denmark. The activity of any of those additional enzymes can be analyzed using any method known in the art for the enzyme in question, including the methods mentioned in the references cited.

In a preferred embodiment, the DNase is added in combination with carbohydrate oxidase. It is surprisingly found that DNase and carbohydrate oxidase have a synergistic effect in preventing a build-up of slime or removing slime from a surface contacted with water from a pulp or paper making process.

According to the present invention, a carbohydrate oxidase (EC 1.1.3) refers to an enzyme which is able to oxidize carbohydrate substrates (e.g., glucose or other sugar or oligomer intermediate) into an organic acid, e.g., gluconic acid, and cellobionic acid. These enzymes are oxidoreductases acting on the CH—OH group of electron donors with oxygen as electron acceptor or alternatively physiological acceptors such as quinones, Cytochrome C, ABTS, etc. also known as carbohydrate dehydrogenases. In an embodiment, the carbohydrate oxidase is an oxidoreductase acting on the CH—OH group of electron donors with oxygen as electron acceptor. Examples of carbohydrate oxidases include malate oxidase (EC 1.1.3.3), glucose oxidase (EC 1.1.3.4), hexose oxidase (EC 1.1.3.5), galactose oxidase (EC 1.1.3.9), pyranose oxidase (EC 1.1.3.10), catechol oxidase (EC 1.1.3.14), sorbose oxidase (EC 1.1.3.11), cellobiose oxidase (EC 1.1.3.25), and mannitol oxidase (EC 1.1.3.40). Preferred oxidases include monosaccharide oxidases, such as, glucose oxidase, hexose oxidase, galactose oxidase and pyranose oxidase.

The carbohydrate oxidase may be derived from any suitable source, e.g., a microorganism, such as, a bacterium, a fungus or a yeast. Examples of carbohydrate oxidases include the carbohydrate oxidases disclosed in WO 95/29996 (Novozymes A/S); WO 99/31990 (Novozymes A/S), WO 97/22257 (Novozymes A/S), WO 00/50606 (Novozymes Biotech), WO 96/40935 (Bioteknologisk Institut), U.S. Pat. No. 6,165,761 (Novozymes A/S), U.S. Pat. No. 5,879,921 (Novozymes A/S), U.S. Pat. No. 4,569,913 (Cetus Corp.), U.S. Pat. No. 4,636,464 (Kyowa Hakko Kogyo Co., Ltd), U.S. Pat. No. 6,498,026 (Hercules Inc.); EP 321811 (Suomen Sokeri); and EP 833563 (Danisco A/S).

In one embodiment, the carbohydrate oxidase comprises or consists of cellobiose oxidase, hexose oxidase, pyranose oxidase, galactose oxidase, and/or glucose oxidase activities. In a preferred embodiment, the carbohydrate oxidase comprises or consists of cellobiose oxidase, pyranose oxidase, galactose oxidase, and/or glucose oxidase activities. In a preferred embodiment, the carbohydrate oxidase comprises or consists of cellobiose oxidase, hexose oxidase, galactose oxidase, and/or glucose oxidase activities. In a preferred embodiment, the carbohydrate oxidase comprises or consists of cellobiose oxidase, hexose oxidase, pyranose oxidase, and/or glucose oxidase activities. In a preferred embodiment, the carbohydrate oxidase comprises or consists of cellobiose oxidase activities. In a preferred embodiment, the carbohydrate oxidase comprises or consists of hexose oxidase activities. In a preferred embodiment, the carbohydrate oxidase comprises or consists of pyranose oxidase activities. In a preferred embodiment, the carbohydrate oxidase comprises or consists of galactose oxidase activities. In a preferred embodiment, the carbohydrate oxidase comprises or consists glucose oxidase activities.

The glucose oxidase may be derived from a strain of Aspergillus or Penicillium, preferably, A. niger, P. notatum, P. amagasakiense or P. vitale. Preferably, the glucose oxidase is an Aspergillus niger glucose oxidase. Other glucose oxidases include the glucose oxidases described in “Methods in Enzymology”, Biomass Part B Glucose Oxidase of Phanerochaete chrysosporium, R. L. Kelley and CA. Reddy (1988), 161, pp. 306-317 and the glucose oxidase Hyderase 15 (Amano Pharmaceutical Co., Ltd.).

Hexose oxidase can be isolated, for example, from marine algal species naturally producing that enzyme. Such species are found in the family Gigartinaceae which belong to the order Gigartinales. Examples of hexose oxidase producing algal species belonging to Gigartinaceae are Chondrus crispus and Iridophycus flaccidum. Also algal species of the order Cryptomeniales are potential sources of hexose oxidase. Hexose oxidases have been isolated from several red algal species such as Iridophycus flaccidum (Bean and Hassid, 1956, J. Biol. Chem., 218:425-436) and Chondrus crispus (Ikawa 1982, Methods Enzymol., 89:145-149). Additionally, the algal species Euthora cristata (Sullivan et al. 1973, Biochemica et Biophysica Acta, 309:11-22) has been shown to produce hexose oxidase. Other potential sources of hexose oxidase include microbial species or land-growing plant species. An example of a plant source for a hexose oxidase is the source disclosed in Bean et al., Journal of Biological Chemistry (1961) 236: 1235-1240, which is capable of oxidizing a broad range of sugars including D-glucose, D-galactose, cellobiose, lactose, maltose, D-2-deoxyglucose, D-mannose, D-glucosamine and D-xylose. Another example of an enzyme having hexose oxidase activity is the carbohydrate oxidase from Malleomyces mallei disclosed by Dowling et al., Journal of Bacteriology (1956) 72:555-560. Another example of a suitable hexose oxidase is the hexose oxidase described in EP 833563.

The pyranose oxidase may be derived, e.g., from a fungus, e.g., a filamentous fungus or a yeast, preferably, a Basidomycete fungus. The pyranose oxidase may be derived from genera belonging to Agaricales, such as Oudemansiella or Mycena, to Aphyllophorales, such as Trametes, e.g. T. hirsuta, T. versicolour, T. gibbosa, T. suaveolens, T. ochracea, T. pubescens, or to Phanerochaete, Lenzites or Peniophora. Pyranose oxidases are of widespread occurrence, but in particular, in Basidiomycete fungi. Pyranose oxidases have also been characterized or isolated, e.g., from the following sources: Peniophora gigantea (Huwig et al., 1994, Journal of Biotechnology 32, 309-315; Huwig et el., 1992, Med. Fac. Landbouww, Univ. Gent, 57/4a, 1749-1753; Danneel et al., 1993, Eur. J. Biochem. 214, 795-802), genera belonging to the Aphyllophorales (Volc et al., 198S, Folia Microbiol. 30, 141-147), Phanerochaete chrysosporium (Volc et al., 1991, Arch. Miro-biol. 156, 297-301, Volc and Eriksson, 1988, Methods Enzymol 161 B, 316-322), Polyporus pinsitus (Ruelius et al., 1968, Biochim. Biophys. Acta, 167, 493-500) and Bierkandera adusta and Phebiopsis gigantea (Huwig et al., 1992, op. cit.). Another example of a pyranose oxidase is the pyranose oxidase described in WO 97/22257, e.g., derived from Trametes, particularly T. hirsuta.

Galactose oxidase enzymes are well-known in the art. An example of a galactose oxidase is the galactose oxidases described in WO 00/50606.

Commercially available carbohydrate oxidases include GRINDAMYL™ (Danisco A/S), Glucose Oxidase HP S100 and Glucose Oxidase HP S120 (Genzyme); Glucose Oxidase-SPDP (Biomeda); Glucose Oxidase, G7141, G 7016, G 6641, G 6125, G 2133, G 6766, G 6891, G 9010, and G 7779 (Sigma-Aldrich); and Galactose Oxidase, G 7907 and G 7400 (Sigma-Aldrich). Galactose oxidase can also be commercially available from Novozymes A/S; Cellobiose oxidase from Fermco Laboratories, Inc. (USA); Galactose Oxidase from Sigma-Aldrich, Pyranose oxidase from Takara Shuzo Co. (Japan); Sorbose oxidase from ICN Pharmaceuticals, Inc (USA), and Glucose Oxidase from Genencor International, Inc. (USA).

In some preferred embodiments, a single type of carbohydrate oxidase may be preferred, e.g., a glucose oxidase, when a single carbohydrate source is involved. In other preferred embodiments, a combination of carbohydrate oxidases will be preferred, e.g., a glucose oxidase and a hexose oxidase. In another preferred embodiment, carbohydrate oxidase having a combination of two or more carbohydrate oxidase activities, e.g., a glucose oxidase activity and a hexose oxidase activity, will be preferred. Preferably, the carbohydrate oxidase is derived from a fungus belonging to the genus Microdochium, preferably the fungus is Microdochium nivale, such as Microdochium nivale as deposited under the deposition no CBS 100236, as described in WO 1999/031990 (Novozymes A/S.), which is hereby incorporated by reference. The Microdochium nivale carbohydrate oxidase has activity on a broad range of carbohydrate substrates. Preferably, the carbohydrate oxidase is derived from a fungus belonging to the genus Aspergillus, preferably the fungus is a strain derived from Aspergillus Niger as described in WO 2017/202887 (Novozymes A/S.), which is hereby incorporated by reference.

In a preferred embodiment, the carbohydrate oxidase has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 6. In one embodiment the mature polypeptide of SEQ ID NO: 6 corresponds the amino acids 23 to 495 of SEQ ID NO: 6.

An example of a lipase is the RESINASE A2X lipase.

Examples of cutinases are those derived from Humicola insolens (U.S. Pat. No. 5,827,719); from a strain of Fusarium, e.g., F. roseum culmorum, or particularly F. solani pisi (WO 90/09446; WO 94/14964, WO 94/03578). The cutinase may also be derived from a strain of Rhizoctonia, e.g., R. solani, or a strain of Alternaria, e.g., A. brassicicola (WO 94/03578), or variants thereof such as those described in WO 00/34450, or WO 01/92502.

Examples of proteases are the ALCALASE, ESPERASE, SAVINASE, NEUTRASE and DURAZYM proteases. Proteases can be derived from Nocardiopsis, Aspergillus, Rhizopus, Bacillus clausii, Bacillus alcalophilus, B. cereus, B. natto, B. vulgatus, B. mycoide, and subtilisins from Bacillus, especially proteases from the species Nocardiopsis sp. and Nocardiopsis dassonvillei such as those disclosed in WO 88/03947, and mutants thereof, e.g. those disclosed in WO 91/00345 and EP 415296.

Specific examples of pectinase that can be used are pectinase AEI, Pectinex 3X, Pectinex 5X and Ultrazyme 100.

Examples of peroxidases and laccases are disclosed in EP 730641; WO 01/98469; EP 719337; EP 765394; EP 767836; EP 763115; and EP 788547.

Examples of cellulases are disclosed in co-pending application US application U.S. 60/941,251, which is hereby incorporated by reference. In an embodiment the cellulase preparation also comprises a cellulase enzymes preparation, preferably the one derived from Trichoderma reesei.

Examples of endoglucanases are the NOVOZYM 613, 342, and 476, and NOVOZYM 51081 enzyme products.

An example of a xylanase is the PULPZYME HC hemicellulase.

Examples of mannanases are the Trichoderma reesei endo-beta-mannanases described in Ståhlbrand et al, J. Biotechnol. 29 (1993), 229-242.

Examples of amylases are the BAN, AQUAZYM, TERMAMYL, and AQUAZYM Ultra amylases.

An Example of glucoamylase is SPIRIZYME PLUS.

Examples of galactanase are from Aspergillus, Humicola, Meripilus, Myceliophthora, or Thermomyces.

Examples of levanases are from Rhodotorula sp.

Surfactants can in one embodiment include poly(alkylene glycol)-based surfactants, ethoxylated dialkylphenols, ethoxylated dialkylphenols, ethoxylated alcohols and/or silicone based surfactants.

Examples of poly(alkylene glycol)-based surfactant are poly(ethylene glycol) alkyl ester, poly(ethylene glycol) alkyl ether, ethylene oxide/propylene oxide homo- and copolymers, or poly(ethylene oxide-co-propylene oxide) alkyl esters or ethers. Other examples include ethoxylated derivatives of primary alcohols, such as dodecanol, secondary alcohois, poly[propylene oxide], derivatives thereof, tridecylalcohol ethoxylated phosphate ester, and the like.

Specific presently preferred anionic surfactant materials useful in the practice of the invention comprise sodium alpha-sulfo methyl laurate, (which may include some alpha-sulfo ethyl laurate) for example as commercially available under the trade name ALPHA-STEP™-ML40; sodium xylene sulfonate, for example as commercially available under the trade name STEPANATE™-X; triethanolammonium lauryl sulfate, for example as commercially available under the trade name STEPANOL™-WAT; diosodium lauryl sulfosuccinate, for example as commercially available under the trade name STEPAN™-Mild SL3; further blends of various anionic surfactants may also be utilized, for example a 50%-50% or a 25%-75% blend of the aforesaid ALPHA-STEP™ and STEPANATE™ materials, or a 20%-80% blend of the aforesaid ALPHA-STEP™ and STEPANOL™ materials (all of the aforesaid commercially available materials may be obtained from Stepan Company, Northfield, Ill.).

Specific presently preferred nonionic surfactant materials useful in the practice of the invention comprise cocodiethanolamide, such as commercially available under trade name NINOL™-11CM; alkyl polyoxyalkylene glycol ethers, such as relatively high molecular weight butyl ethylenoxide-propylenoxide block copolymers commercially available under the trade name TOXIMUL™-8320 from the Stepan Company. Additional alkyl polyoxyalkylene glycol ethers may be selected, for example, as disclosed in U.S. Pat. No. 3,078,315. Blends of the various nonionic surfactants may also be utilized, for example a 50%-50% or a 25%-75% blend of the aforesaid NINOL™ and TOXIMUL™ materials.

Specific presently preferred anionic/nonionic surfactant blends useful in the practice of the invention include various mixtures of the above materials, for example a 50%-50% blends of the aforesaid ALPHA-STEP™ and NINOL™ materials or a 25%-75% blend of the aforesaid STEPANATE™ and TOXIMUL™ materials.

Preferably, the various anionic, nonionic and anionic/nonionic surfactant blends utilized in the practice of the invention have a solids or actives content up to about 100% by weight and preferably have an active content ranging from about 10% to about 80%. Of course, other blends or other solids (active) content may also be utilized and these anionic surfactants, nonionic surfactants, and mixtures thereof may also be utilized with known pulping chemicals such as, for example, anthraquinone and derivatives thereof and/or other typical paper chemicals, such as caustics, defoamers and the like.

The method of the present invention is an efficient and environmentally friendly way to prevent a build-up of slime or remove slime from a surface contacted with water. In a preferred embodiment, the method of the present invention can further reduce downtime by avoiding the need of cleaning or breaks in the pulp or paper making process; reduce spots or holes in a final product; reduce spores in a final product; or reduce blocking of devices such as filters or wires or nozzles, or partly or totally replace biocides. In another preferred embodiment, the method of the present invention can reduce downtime by avoiding the need of cleaning or breaks in the pulp or paper making process. Cleaning stops or breaks and the corresponding downtime are the most common runnability problems in a pulp or paper making mill. By reducing cleaning time and the amount of breaks the method of the present invention will increase production. In another preferred embodiment, the method of the present invention can reduce spots or holes in a final product. Quality of paper or paperboard is affected by sheet defects from microbiological deposition. By controlling the slime, the method of the present invention effectively reduces spots or holes in a final product. In another preferred embodiment, the method of the present invention can reduce blocking of devices such as filters or wires or nozzles. Slime can block devices such as filters or wires or nozzles. By controlling slime, the method of the present invention effectively reduces blocking of devices such as filter or wires or nozzles. In another preferred embodiment, the method of the present invention allows a partial or total reduction on the use of conventional biocides. The method of present invention provides a greener alternative to toxic biocides which are needed by the pulp and paper industry.

It was found that the method of the present invention has a highly superior effect in the control of slime when compared to the commercial benchmark protease. At the same protein dosage, the prevention effect of the DNase is improved by about 10-5000%, preferably 15-3000%, more preferably 20-2000% compared to the one achieved by the best-in-class protease.

In another aspect, the present invention relates to a method of preventing a build-up of slime or removing slime from a surface contacted with water from a pulp or paper making process, comprising the steps of
(a) preparing a composition comprising a DNase; and
(b) adding the composition to the water from a pulp or paper making process.

In another aspect, the present invention provides a method of manufacturing pulp or paper, comprising subjecting water from pulp or paper manufacturing process to a DNase. In an embodiment, the method of present invention prevents the build-up of slime or removes slime from a surface contacted with water from a pulp or paper making process. In another embodiment, the method of the present invention controls or reduces odour from a pulp or paper making process.

In another aspect, the present invention provides use of a DNase in preventing the build-up of slime or removing slime from a surface contacted with water from a pulp or paper manufacturing process.

In a preferred embodiment, the water is cleaning water, process water, wastewater, and/or water mist in the air. In another preferred embodiment, the DNase has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 1, SEQ ID NO: 2, the mature polypeptide of SEQ ID NO: 3, the mature polypeptide of SEQ ID NO: 4, or the mature polypeptide of SEQ ID NO: 5.

In another aspect, the present invention relates to a composition for preventing a build-up of slime or removing slime from a surface contacted with water from a pulp or paper making process, comprising a DNase and an additional enzyme; a DNase and a surfactant; or a DNase, an additional enzyme and a surfactant. In one embodiment, the composition comprises a DNase and a carbohydrate oxidase. In another embodiment, the composition comprises a DNase, a carbohydrate oxidase and a surfactant.

Any enzyme having carbohydrate oxidase, lipase, cutinase, protease, pectinase, laccase, peroxidase, cellulase, glucanase, xylanase, mannanase, lysozyme, amylase, glucoamylase, galactanase, and/or levanase activities can be used as additional enzymes in the composition of the invention.

Various anionic, nonionic and anionic/nonionic surfactant can be used as the surfactant in the composition of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

### Materials and Methods

Chemicals used as buffers and substrates were commercial products of at least reagent grade.

The process waters from the industrial papermaking process were sampled in the water circulation loop of the paper machine. They were stored in a refrigerated room at ca. 5° C. and used as described in the examples.

Specific enzymes used in the examples:

variant,

| DNase-1 | DNase variant, | DNase variant of SEQ ID NO: 1 herein with |
|---|---|---|
| | prepared according to | substitutions T1I + S13Y + T22P + S27L + |
| | WO 2019/081724 | L33K + S39P + S42G + D56I + S57W + |
| | | S59V + T65V + V76L + Q109R + S116D + |
| | | T127V + S144P + A147H + S167L + |
| | | G175D |
| DNase-2 | DNase variant | DNase variant of SEQ ID NO: 2 herein with |
| | prepared according to | substitutions N61D + T65I + S82R + K107Q |
| | WO 2019/081724 and | |
| | EP 21162531.4 | |
| DNase-3 | DNase variant, | DNase variant of SEQ ID NO: 2 herein with |
| | prepared according to | substitutions T65V + G149N |
| | WO 2019/081724 and | |
| | EP 21162531.4 | |
| DNase-4 | DNase variant, | DNase variant of SEQ ID NO: 2 herein with |
| | prepared according to | substitutions N61D + T65I + K107Q + |
| | WO 2019/081724 and | T127S + S164D |
| | EP 21162531.4 | |
| DNase-5 | DNase derived from | mature polypeptide of DNase shown in SEQ |
| | Morchella costata, | ID NO: 3 herein |
| | prepared according to | |
| | SEQ ID NO: 11 of | |
| | WO2018177203 | |
| DNase-6 | DNase derived from | mature polypeptide of DNase shown in SEQ |
| | Urnula sp-56769, | ID NO: 4 herein |
| | prepared according to | |
| | SEQ ID NO: 5 of | |
| | WO2018177203 | |
| DNase-7 | DNase from | mature polypeptide of DNase shown in SEQ |
| | Neosartorya massa | ID NO: 5 herein |
| | SEQ ID NO: 184 of | |
| | WO2017059802 | |
| Carbohydrate | Carbohydrate oxidase | mature polypeptide of SEQ ID NO: 6 herein |
| oxidase | derived from | |
| | Microdochium nivale, | |
| | prepared according to | |
| | SEQ ID NO: 2 of WO | |
| | 1999/031990 | |
| Protease | a Bacillus clausii | SEQ ID NO: 7 herein |
| | protease, prepared | |
| | according to SEQ ID | |
| | NO: 1 in WO | |
| | 2011/036263 | |

### Process Water Samples Used in the Examples:

| Process | | | Conductivity | Redox potential | Cellular ATP *⁾ |
|---|---|---|---|---|---|
| water ID | Origin | pH | (μS/cm) | (mV) | (ng/mL) |
| PW1 | Recycled | 6.9 | 3120 | −40 | 229 |
| | packaging board | | | | |
| | production | | | | |
| PW2 | Recycled | 7.2 | 4460 | −8 | 517 |
| | packaging board | | | | |
| | production | | | | |

*⁾ Cellular ATP, Adenosine Triphosphate, was measured with LuminUltra test kit QuenchGone21 Industrial (QG21I ™).

### Example 1

Measurement of Slime Prevention Effect by a DNase Using Process Water from Recycled Packaging Board Process

A sample of process water, PW1, from the paper machine water loop from an industrial production of recycled packaging board was used as microbiol inoculum for the slime cultivation experiments in a micro-titer plate (MTP; 96 wells; Thermo Scientific Nunc Edge microwell 96F well plate, clear, with lid, Sterile). This process water was mixed with a buffer (800 mM MES pH 6.8) in 85:15 volume proportion, and 130 μL was added to each MTP well followed by the addition of 20 μL of diluted enzyme or sterilized RO water (control—without enzyme). The MTP plate was incubated at 40° C. for 96 hours in an incubator (Heraeus B 6120). Each column of the MTP plate corresponds to a different treatment (control vs. enzyme) done in six wells. The enzymes were diluted to target concentration in the final volume (150 μL) in 20 mM sterilized RO water.

After the incubation time, the solution was discarded from the MTP plates and the wells were gently washed with 300 μL of 0.9% NaCl solution in one step. After discarding the washing solution the slime was fixated at 60° C. for 30 min in an benchtop orbital shaker (Thermo Scientific, MaxQ 4450) and was allowed to cool before 150 μL of 0.095% crystal violet (CAS No. 548-62-9) solution was added to the wells and left for 15 mins to stain the slime that was formed. The crystal violet solution was then discarded and 300 μL of 0.9% NaCl solution was gently added to the wells in two consecutive steps while discarding the washing solution after each washing step. Finally, 150 μL of 40% acetic acid was added and let it to react for 20 min. The amount of color released from the slime was measured by the Absorbance (ABS) at 600 nm in a spectrophotometer (SpectraMax plus 384) and was used to quantify the amount of slime that was produced on the plastic surface. Average of 6 ABS measurements of all samples (outliers excluded according to the Median Absolute Deviation method) was used to calculate the resulting % of slime reduction of each enzyme treatment in relation to the control according to the below formula. The Blank was measured as being the ABS of nutrient medium without process water. If more than one control was present in the MTP (i.e., more than one column for the same sample), the average of the corresponding number of wells was calculated.

$$Slime reduction ( \% ) = \frac{( {ABS}_{Control} - {ABS}_{Blank} ) - ( {ABS}_{Treatment} - {ABS}_{Blank} )}{( {ABS}_{Control} - {ABS}_{Blank} )} * 100 \%$$

It is seen in Table 1 that the DNase-1 achieved the best slime prevention effect ranging from 35% to 76% versus the commercial benchmark protease ranging from 0 to 59% with the same enzyme protein dosage range applied. A clear dosage response was observed for both treatments, where the DNase-1 outperformed the commercial benchmark protease at all enzyme protein concentrations, showing improvements versus the protease from 1149% to 30% depending on the actual enzyme protein dosage.

TABLE 1

| | Enzyme | | | Relative improvement in |
|---|---|---|---|---|
| | protein (EP) | | Slime | slime reduction by DNase |
| | dosage (mg | ABS at | reduc- | versus protease at same |
| Treatment | EP/L) | 600 nm | tion | EP dosage |
| Control | 0 | 0.834 | — | — |
| Protease | 10 | 0.849 | 0 | — |
| Protease | 20 | 0.796 | 5% | — |
| Protease | 30 | 0.748 | 11% | — |
| Protease | 40 | 0.509 | 43% | — |
| Protease | 60 | 0.387 | 59% | — |
| DNase-1 | 10 | 0.570 | 35% | — |
| DNase-1 | 20 | 0.356 | 63% | 1149% |
| DNase-1 | 30 | 0.305 | 70% | 513% |
| DNase-1 | 40 | 0.284 | 72% | 69% |
| DNase-1 | 60 | 0.253 | 76% | 30% |
| Blank | 0 | 0.073 | — | — |

### Example 2

Measurement of Slime Prevention Effect by a DNase Using Process Water from Recycled Packaging Board Process

A sample of process water, PW1, from the paper machine water loop from an industrial production of recycled packaging board was used as microbiol inoculum for the slime cultivation experiments in a micro-titer plate as described in Example 1. The effect of alternative DNases was tested.

Incubation, measurement of absorbance and calculation of slime reduction was performed as described in Example 1.

Six different DNases had slime prevention properties as seen in Table 2. DNase-2 gave a much higher slime reduction effect than the benchmark protease (80% reduction versus 58% reduction). DNases-3 and -4 had a comparable performance to the protease, and DNases-5, -6 and -7 had a slightly lower performance than the protease.

TABLE 2

| | Enzyme protein | | |
|---|---|---|---|
| | (EP) dosage | ABS at | Slime |
| Treatment | (mg EP/L) | 600 nm | reduction |
| Control | 0 | 0.642 | — |
| Protease | 80 | 0.307 | 58% |
| DNase-2 | 80 | 0.195 | 80% |
| DNase-3 | 80 | 0.311 | 58% |
| DNase-4 | 80 | 0.346 | 54% |
| DNase-5 | 80 | 0.460 | 34% |
| DNase-6 | 80 | 0.542 | 20% |
| DNase-7 | 80 | 0.569 | 15% |
| Blank | 0 | 0.076 | — |

### Example 3

Measurement of Slime Prevention Effect by Combination of a DNase and Carbohydrate Oxidase Using Process Water from Recycled Packaging Board Process

Samples of process water, PW1 and PW2, from the paper machine water loop from an industrial production of recycled packaging board was used as microbiol inoculum for the slime cultivation experiments in a micro-titer plate as described in Example 1. The effect of combining a DNase and a carbohydrate oxidase was tested by adding both enzymes to the same wells in the micro-titer plate. The slime prevention effect of combined enzyme treatment was compared to wells with single enzyme treatment, where either the DNase or the carbohydrate oxidase was added alone.

Incubation, measurement of absorbance and calculation of slime reduction was performed as described in Example 1. The improved performance achieved by combining the DNase or the carbohydrate oxidase was calculated according to the formula given below.

$$Improved performance ( \% ) = \frac{Slime reduction \% ( combined ) - Slime reduction \% ( addition )}{Slime reduction \% ( addition )} * 100 \%$$

where
Slime reduction % (combined) is the measured slime reduction, when the DNase (dosage X) and the carbohydrate oxidase (dosage X) were added to the same well,
and
Slime reduction % (addition) is the sum of the measured slime reduction of the DNase (dosage X) added alone and the carbohydrate oxidase (dosage X) added alone.

The combined treatment with DNase-1 and carbohydrate oxidase gave a higher slime reduction % compared to the added effects of single enzyme treatment as seen in Table 3 and 4. Synergistic effects of combining DNase-1 and carbohydrate oxidase was observed at enzyme dosages 15+15 mg EP/L, where 13% improved performance was found, at 10+10 mg EP/L, where 81% improved performance was found, and at 5+5 mg EP/L, where 14% improved performance was found. Synergy was clearly seen with both tested process waters, PW1 and PW2 (Table 3 and 4 respectively).

TABLE 3
Results obtained with process water PW1

| | Enzyme protein (EP) dosage | | | | |
|---|---|---|---|---|---|
| | (mg EP/L) | | | | |
| | | Carbohydrate | ABS at | Slime | Improved |
| Treatment | DNase-1 | oxidase | 600 nm | reduction | performance |
| Control | — | — | 0.834 | — | — |
| DNase-1 | 15 | — | 0.622 | 28% | — |
| Carbohydrate oxidase | — | 15 | 0.704 | 17% | — |
| DNase-1 + | 15 | 15 | 0.447 | 50% | 13% |
| Carbohydrate oxidase | | | | | |
| DNase-1 | 10 | — | 0.668 | 22% | — |
| Carbohydrate oxidase | — | 10 | 0.934 | 0% | — |
| DNase-1 + | 10 | 10 | 0.528 | 40% | 81% |
| Carbohydrate oxidase | | | | | |
| Blank | — | — | 0.067 | — | — |

TABLE 4
Results obtained with process water PW2

| | Enzyme protein (EP) dosage | | | | |
|---|---|---|---|---|---|
| | (mg EP/L) | | | | |
| | | Carbohydrate | ABS at | Slime | Improved |
| Treatment | DNase-1 | oxidase | 600 nm | reduction | performance |
| Control | — | — | 0.877 | — | — |
| DNase-1 | 5 | — | 0.738 | 17% | — |
| Carbohydrate oxidase | — | 5 | 0.782 | 12% | — |
| DNase-1 + | 5 | 5 | 0.612 | 33% | 14% |
| Carbohydrate oxidase | | | | | |
| Blank | — | — | 0.067 | — | — |

1-19. (canceled)

20: A method of preventing a build-up of slime or removing slime from a surface contacted with water from a pulp or paper making process, the method comprising contacting said water with a DNase.

21: The method according to claim 20, wherein the DNase has at least 80%, sequence identity to SEQ ID NO: 1, SEQ ID NO: 2, the mature polypeptide of SEQ ID NO: 3, the mature polypeptide of SEQ ID NO: 4, or the mature polypeptide of SEQ ID NO: 5.

22: The method according to claim 20, wherein the DNase is a DNase variant, which compared to a DNase with SEQ ID NO: 1, comprises one or more substitutions selected from the group consisting of: T1I, T1L, T1V, S13Y, T22P, S25P, S27L, S39P, S42G, S42A, S42T, S57W, S57Y, S57F, S59V, S591, S59L, V76L, V761, Q109R, S116D, S116E, T127V, T1271, T127L, S144P, A147H, S167L, S1671, S167V, G175D and G175E;
wherein the positions correspond to the positions of SEQ ID NO: 1 (numbering according to SEQ ID NO: 1);
wherein the variant has a sequence identity to the polypeptide shown in SEQ ID NO: 1 of at least 80%; and
wherein the variant has a DNase activity.

23: The method according to claim 22, wherein the DNase variant comprises a combination of substitutions selected from the group consisting of:
T1I+S13Y+T22P+S25P+S27L+S39P+S42G+S57W+S59V+V76L+S144P+A147H+S167L+G 175D;
T1I+S13Y+T22P+S27L+S42G+S57W+S59V+V76L+Q109R+S116D+T127V+S144P+A147 H+S167L+G175D;
T1I+S13Y+T22P+S25P+S27L+S39P+S42G+S57W+S59V+V76L+Q109R+S116D+T127V+S144P+A147H+S167L+G175D;
T1I+S13Y+T22P+S25P+S27L+S39P+S42G+S57W+S59V+V76L+T77Y+Q109R+S116D+T 127V+S144P+A147H+S167L+G175D;
T1I+T22P+D561+S57W+V76L+Q109R+S116D+A147H+G162S+S167L+G175N+N178;
T1I+S13Y+T22P+S27L+S39P+S42G+D561+S57W+S59V+V76L+Q109R+S116D+T127V+S 144P+A147H+S167L+G175D;
T1I+T22P+S25P+S27L+S42G+D561+S57Y+S59V+V76L+T77Y+Q109R+S116D+T127V+S1 44P+A147H+Q166D+S167L+G175D+S181L;
T1I+S13Y+T22P+S25P+S27L+S39P+S42G+D561+S57W+S59V+V76L+Q109R+S116D+T1 27V+S144P+A147H+S167L+G175D;
T1I+S13Y+T22P+S25P+S27L+S39P+S42G+D561+S57W+S59V+V76L+Q109R+S116D+T1 27V+S144P+A147H+Q166D+S167L+G175D;
T1I+S13Y+T22P+S27R+S39P+S42G+D561+S57W+S59V+V76L+Q109R+S116D+T127V+S 144P+A147H+S167L+G175D;
T1I+S13Y+T22P+S27L+S39P+S42G+D561+S57W+S59V+T65L+V76L+Q109R+S116D+T1 27V+S144P+A147H+S167L+G175D;
T1I+S13Y+T22P+S27L+L33K+S39P+S42G+D561+S57W+S59V+T65V+V76L+Q109R+S11 6D+T127V+S144P+A147H+S167L+G175D;
T1I+S13Y+T22P+S25P+S27R+S39P+S42G+S57W+S59V+S66W+V76L+T77Y+Q109R+S1 16D+T127V+S144P+A147H+S167L+G175D;
T1I+S13Y+T22P+S25P+S27L+L33K+S39P+S42G+S57W+S59V+T65V+V76L+T77Y+Q109 R+S116D+T127V+S144P+A147H+S167L+G175D;
T1I+S13Y+T22P+S25P+S27L+L33K+S39P+S42G+S57W+S59V+S66Y+V76L+T77Y+Q109 R+S116D+T127V+S144P+A147H+S167L+G175D;
T1I+S13Y+T22P+S25P+S27L+S39P+S42G+S57W+S59V+T65V+S66Y+V76L+T77Y+Q109 R+S116D+T127V+S144P+A147H+S167L+G175D;
T1I+S13Y+T22P+S27L+S39P+S42G+D56L+S57W+S59V+T65V+V76L+Q109R+S116D+T1 27V+S144P+A147H+G162D+S167L+G175D;
T1I+S13Y+T22P+S27R+S39P+S42G+D56L+S57W+S59V+T65V+V76L+Q109R+S116D+T 127V+S144P+A147H+S167L+G175D;
T1I+S13Y+T22P+S27R+S39P+S42G+D56L+S57W+S59V+T65V+V76L+Q109R+S116D+T 127V+S144P+A147H+G162D+S167L+G175D;
T1I+S13Y+T22P+S27K+S39P+S42G+D561+S57W+S59V+T65V+V76L+S106L+Q109R+S1 16D+T127V+S144P+A147H+S167L+G175D;
T1I+S13Y+T22P+S27K+S39P+S42G+D561+S57W+S59V+T65V+V76L+Q109R+S116D+T1 27V+S130A+S144P+A147H+S167L+G175D;
T1I+S13Y+T22P+S27L+S39P+S42G+D56L+S57W+S59V+T65L+V76L+Q109R+S116D+T1 27V+S130A+S144P+A147H+S167L+G175D; and
T1I+S13Y+T22P+S27L+S39P+S42G+D561+S57W+S59V+T65V+V76L+Q109R+S116D+T1 27V+S144P+A147H+G162D+S167L+G175.

24: The method of claim 20, wherein the DNase is a DNase variant, which compared to the polypeptide of SEQ ID NO: 2 comprises one or more substitutions selected from the group consisting of N61D, T651, T65V, S82R, K107Q, T127S, T127V, G149N, S164D and L181S;
wherein the variant has at least 80% sequence identity to SEQ ID NO: 2; and
wherein the variant has DNase activity.

25: The method of claim 24, wherein the DNase variant comprises a combination of substitutions selected from the group consisting of:
K21 L+Q48D+T651+S82R+K107Q+T127S;
Q14R+K21 L+Q48D+T651+T127S;
Q48D+T651+S82R+T127S+S164D;
N61D+T651+K107Q+T127S+S164D;
Q48D+T651+S82R+K107Q+T127S;
Q14R+N61D+T651+S82R+K107Q;
N61D+S68L+G149N;
Q14R+N61D+T651+S82R+T127S+S164D;
K21L+Q48D+T651+S82R+K107Q;
Q14R+T651+K107Q+T127S;
N61D+T651+S82R+T127S+S164D;
K21 L+N61D+T651+S82R+K107Q+T127S;
T65V+T127V+G149N;
T651+K107Q+T127S+S164D;
N61D+T651+S82R+K107Q;
Q14R+K21 L+N61D+T651+S82R;
Q14R+K21 L+N61D+T651+S82R+K107Q;
Q14R+K21 L+N61D+T651+T127S;
N61 D+T651+S82R+K107Q+T127S+S164D;
Q14R+K21L+T651+K107Q+T127S;
N61 D+T651+K107Q+T127S;
T651+S82R+K107Q+S164D;
K21L+N61 D+T651+S82R;
K21L+N61 D+T651+T127S;
N61 D+T651+S82R+S164D;
K21L+N61 D+T651+S82R+K107Q;
S68L+S106A+G149N;
N61 D+T651+T127S+S164D;
Q14R+K21L+N61 D+T651;
Q14R+K21L+T651+T127S;
T65V+G149N;
T65V+R109T+T127V;
Q14R+K21L+T651+K107Q;
K21L+T651+S82R+K107Q;
K21L+T651+K107Q+T127S;
N61D+S68L+S102Y+G149N+S164D+L181T;
N61 D+S68L+S106A+G149N+S164D;
T65V+R109T+G149N;
T65V+T127V+T171W;
T65V+T127V+L181S;
T651+S164D+L181W;
N61 D+S66Y+S102Y+S164D;
N61 D+S66Y+S164D;
N61 D+T65V+S164D;
Q14W+N61 D+T651;
Q14W+N61 D+T651+S164D;
Q14W+N61 D+T651+S164D+L181W;
T651+D116W+S164D+L181W;
T651+D116W+S164D;
Q14W+T651+S164D;
R109T+G149N;
G149N+T171W;
P25S+L33K+D561+T65V+Y77T+T127V+L181S;
P25S+L33K+T65V+Y77T+T127V+L181S;
P25S+L33K+D561+T65V+Y77T+R109Q+T127V+L181S;
P25S+L33K+D561+T65V+Y77T+D116S+T127V+L181S;
D56L+T65V+T127V;
D56L+T65V+T127V+T171W;
T65V+G149N+T171W;
Q48D+T651+K107Q+T127S+S164D;
T65V+Y77T+T127V;
T65V+R109T+T127V+G149N;
T65V+R109T+T171W;
T65V+R109T+G149N+T171W;
P25S+D561+T65V+Y77T+T127V+L181S;
Q14R+K21L+T651+K107Q+T127S;
N61 D+S68L+S102Y;
S66Y+T127V+L181S;
N61 D+T651+S82R+K107Q+L181 D;
T65V+G149N+L181E;
T65V+T127V+G149N+Y182D;
Q48D+N61 D+T651+S82R+K107Q;
Q48D+T65V+G149N;
N61 D+T651+S82R+T127S+S164D+Y182D;
N61 D+T651+S82R+T127S+S164D+L181 D;
Q48D+N61 D+T651+K107Q+T127S+S164D;
N61 D+T651+S82R+T127S+S164D+Y182N;
T651+K107Q+T127S+S164D+Y182D;
N61 D+T651+K107Q+T127S+S164D+L181E;
N61 D+T651+K107Q+T127S+S164D+L181 D;
T651+K107Q+T127S+S164D+L181T;
T651+K107Q+T127S+S164D+L181E;
N61 D+T651+K107Q+T127S+S164D+Y182D;
T651+K107Q+T127S+S164D+Y182N;
N61 D+T651+K107Q+T127S+S164D+L181Q;
N61 D+T65V+S164D+Y182N;
N61 D+T651+K107Q+T127S+S164D+L181T;
T651+K107Q+T127S+S164D+L181 D;
T651+K107Q+T127S+S164D+L181Q;
N61 D+T65V+T127S+S164D;
Q48D+N61 D+T65V+S164D;
K21L+N61 D+T651+K107Q+T127S;
Q14W+N61 D+T651+R109T+G149N+S164D+L181W;
K21L+N61 D+T651+K107Q;
Q14W+N61 D+T651+R109T+G149N;
Q14W+T65V+R109T+G149N+W1541+L181W;
Q14W+T65V+R109T+G149N+W1541+S164D;
Q14W+N61 D+T65V+R109T+G149N+L181W;
Q14W+T651+R109T+D116W+G149N+S164D+L181W;
T65V+R109T+T127V+T171W; and
R109T+T127V+T171W.

26: The method according to claim 20, wherein the DNase is added in an amount of 0.001-1000 mg enzyme protein/L.

27: The method according to claim 20, wherein the DNase is added in an amount of 0.1-10 mg enzyme protein/L.

28: The method according to claim 20, wherein the water is cleaning water, process water, wastewater, and/or water mist in the air.

29: The method according to claim 20, wherein the water has pH from 4 to 10, conductivity from 100 μS/cm to 12000 μS/cm, redox potential from −500 mV to 1500 mV and/or cellular ATP from 0.1 ng/ml to 1000 ng/ml.

30: The method according to claim 20, wherein the water has pH from 6.1 to 7.6, conductivity from 1772 μS/cm to 5620 μS/cm, redox potential from −110 mV to 210 mV and/or cellular ATP from 4.2 ng/ml to 114 ng/ml.

31: The method according to claim 20, wherein the surface is a plastic surface or a metal surface.

32: The method according to claim 20, wherein the surface is the surface from a manufacturing equipment.

33: The method according to claim 20, wherein the pulp or paper making process is a process for manufacturing paper or packaging material.

34: The method according to claim 20, further comprising contacting said water with one or more enzymes selected from the group consisting of carbohydrate oxidase, lipase, cutinase, protease, pectinase, laccase, peroxidase, cellulase, glucanase, xylanase, mannanase, lysozyme, amylase, glucoamylase, galactanase, and levanase.

35: The method according to claim 34, wherein the enzyme is a carbohydrate oxidase having at least 80% sequence identity to the mature polypeptide of SEQ ID NO: 6.

36: A method of preventing a build-up of slime or removing slime from a surface contacted with water from a pulp or paper making process, the method comprising the steps of
(a) preparing a composition comprising a DNase; and
(b) adding the composition to the water from a pulp or paper making process.

37: A method of manufacturing pulp or paper, comprising subjecting water from pulp or paper making process to a DNase.

38: A composition for preventing a build-up of slime or removing slime from a surface contacted with water from a pulp or paper making process, comprising a DNase and an additional enzyme; a DNase and a surfactant; or a DNase, an additional enzyme and a surfactant.

39: The composition of claim 38, comprising a DNase and carbohydrate oxidase; or a DNase, carbohydrate oxidase and a surfactant.
